(19) United States
(12) Patent Application Publication
(10) Pub. No.: US 2023/0162226 A1
(43) Pub. Date: May 25, 2023

(54) SYSTEM, SERVER AND METHOD FOR HEALTH ACTIVITY ANALYSES AND FINANCIAL REWARDS INTEGRATION

(71) Applicant: Tam(squared) Inc., Wellesley, MA (US)

(72) Inventors: Joel Anthony Lieginger, Mill Valley, CA (US); Anil Trevor Lodhia, Oakland, CA (US); John Martin Hann, Dunstable, MA (US); Elizabeth Coleman Cook, Austin, TX (US); Jessica Dillon Bieligk, Washington, DC (US); Alexis Sean Abril, Dallas, TX (US)

(21) Appl. No.: 17/992,441

(22) Filed: Nov. 22, 2022

Related U.S. Application Data
(60) Provisional application No. 63/282,005, filed on Nov. 22, 2021.

(51) Int. Cl. G06Q 30/0234; G16H 20/30; G06Q 20/36; G06Q 20/10; G06Q 30/0214

(52) U.S. Cl. CPC G06Q 30/0234; G16H 20/30; G06Q 20/36; G06Q 20/10; G06Q 30/0214

(57) ABSTRACT

The disclosure is direct to a system to link rewards to health related activities. In some embodiments, the system is configured to enable a user to link health monitoring software to sensors that monitor health related activities. In some embodiments, the system is configured enable multiple users to link to each other's health monitoring software and obtain rewards based on each other's performance. In some embodiments, the system is configured to automatically calculate reward values based on milestones or goals. In some embodiments, the system is configured to adjust values in third party systems based on a user achieving milestones or goals.

### CROSS-REFERENCE TO RELATED APPLICATION

This application claims the benefit of and priority to U.S. Provisional Application No. 63/282,005, filed Nov. 22, 2021, which is incorporated herein by reference in its entirety.

### BACKGROUND

It is difficult for many to find the motivation to maintain an effective wellness regime. It can take months and even years to achieve one's goals. A major reason for this extended time period is that financial and other concerns often cause people to lose focus on being healthy. We're constantly told to exercise often, eat better, sleep more, and prioritize mental health. The information overload for how to achieve these goals is endless but ignores a key element of true holistic health—financial wellbeing. Health is a financial asset—the more you invest in your overall health, the higher your return on life. Healthier people are often more productive members of society, but currently there isn't a system that connects finances to mental and physical wellbeing to improve all three.

More than 80% of disease burden and 70% of death worldwide is driven by preventable lifestyle behaviors which can often be offset by regular exercise, good nutrition, enough sleep, and mental clarity. Meanwhile, much of the industry financial underwriting, by default, creates moral hazard and adverse selection by pooling the disease and death risk of those who do and do not maintain healthy lifestyles, resulting in overpriced premiums and providing an incentive for individuals to misreport their information during the process or discontinue beneficial activities thereafter.

As it pertains specifically to fitness, global health authorities recommend that adults should perform at least 150 minutes a week of moderate-intensity exercise, such as a brisk walk, or 75 minutes a week of vigorous-intensity aerobic physical activity, such as a run, or an equivalent combination of moderate- and vigorous-intensity aerobic activity. At least two of the days should also include anaerobic activity such as weightlifting.

Studies have found, however, that only 23.2% of American adults (aged 18 and over) meet the recommendation for both aerobic and muscle-strengthening activities. Moreover, additional health benefits are also realized with 300 minutes of moderate-intensity exercise, 150 minutes of vigorous-intensity exercise, or a combination of both.

Society treats wellness in a fragmented and misaligned way, across multiple industries including finance and health. Wellness, however, should serve as the overall umbrella, with consumer needs across various industry verticals and a focus on an ongoing lifestyle rather than a point in time, such as a single healthcare-related visit for physical health or a one-time underwriting outcome for a financial product.

Therefore, there is a need in the art for a system and method that bridges the gaps between mental, physical, and financial wellbeing.

### SUMMARY

In some embodiments, the disclosure is directed to servers, systems and methods that provide improved health activity tracking software combined with financial incentives to improve the health of individuals and society. In some embodiments, the system is configured to integrate technology, financial services, and health and wellness to track and reward users for achieving their wellness goals. In some embodiments, the system includes a unified wellness platform that allows multiple users to motivate each other toward achievable goals that improve their well-being. In some embodiments, the system includes wearable devices (e.g., smart watches, GPS monitors, etc.) configured to receive, send, and/or store a user's health activity. In some embodiments, the system includes the use of artificial intelligence to improve health and wellness analytics and determine and collect the most profitable financial incentives. In some embodiments, the system includes a credit system configured to provide financial resources as well as a mechanism to gain financial rewards.

In some embodiments, the system includes an API-driven platform configured to collect health, financial, and community data, and drive action to facilitate a wellness lifestyle through commercialization, personalization, and socialization that creates a symbiotic relationship across realms and drives value at scale. In some embodiments, the system is configured to serve as a system of accountability by incentivizing ongoing and new healthy behaviors while reducing the likelihood of moral hazard and adverse selection.

In some embodiments, the system is configured to enable users to link wearable devices, health profiles, and financial products through a user's profile in a platform. In some embodiments, the platform is a mobile application. In some embodiments, the system is configured to collect health activity as it occurs, such as 150+ weekly elevated heart rate minutes as a non-limiting example. In some embodiments, the system includes a logic engine configured to dynamically reward a user with goods, services, money, cash-back on credit purchases, targeted donations, and discounts based upon tracked characteristics such as previous purchases and favorite workouts as non-limiting examples. In some embodiments, the platform is configured to enable the user to denote intent and engage in a rewards marketplace. In some embodiments, the system is configured to enable merchants or other parties to receive aggregated and unique insights based on users who selected their reward. In some embodiments, the system is configured to directly deposit earned currency into a user currency account (e.g., bank, digital wallet, crypto, etc.).

In some embodiments, the system is configured to automate multiple steps required for a user to procure a credit card. In some embodiments, the system comprises an orchestration layer that is configured to execute one or more of know-your-customer checks, soft and hard credit checks, creating a ledger with the bank, delivering a virtual card, and shipping a physical card (e.g., plastic, metal, etc.) according to some embodiments. In some embodiments, the orchestration layer is configured to enable a user to apply for a credit card, be approved, spend, and/or receive rewards from this financial product within seconds. Additionally, the orchestration layer is configured to account for edge cases (e.g. failures at various stages). In some embodiments, the orchestration layer is configured to execute one or more steps that include verifying users accounts, sending ACH payments to a bank, providing an interface for call-in support, and providing an interface to change a name, make payment, activate card, and/or perform any conventional financial transaction.

In some embodiments, the system is configured to generate an graphical user interface to enable a user to meet with a financial advisor under a system provided membership program to discuss a variety of topics under the unified wellness umbrella platform, including asset management, mortgages, deposit accounts, savings accounts, as well as debit, credit, and charge cards. In some embodiments, the system is configured to provide an initial cash back reward that is enhanced by an additional cash back bonus once certain goals are achieved.

In some embodiments, the system is configured to allow the platform products (including credit and debit cards, insurance, mortgages, and asset management) to be embedded into another party's application, such as an external insurance or financial institution's product as well as a public or private organization's benefits offering. In some embodiments, in the external solutions, a user is able to complete and/or validate unified wellness activities and/or receive rewards and financial products on behalf of the platform.

In some embodiments, the disclosure is directed to a system (also referred to as Paceline herein) that includes health monitoring software and one or more computers comprising one or more processors and one or more computer readable media. In some embodiments, the one or more computer readable media include instructions stored thereon that when executed configure the one or more computers to execute one or more steps. In some embodiments, the one or more steps includes a step to receive, by the one or more processors, a request for a link to the health monitoring software. In some embodiments, the one or more steps includes a step to receive, by the one or more processors, one or more measurements from one or more sensors electronically coupled to the health monitoring software. In some embodiments, the one or more steps includes a step to compare, by the one or more processors, the one or more measurements to one or more wellness milestones. In some embodiments, the one or more steps includes a step to execute, by the one or more processors, a rewards calculation comprising one or more rewards associated the one or more wellness milestones. In some embodiments, the one or more steps includes a step to send, by the one or more processors, the one or more rewards to the health monitoring software.

In some embodiments, the health monitoring software comprises financial software. In some embodiments, the one or more rewards include one or more financial rewards. In some embodiments, the one or more financial rewards include one or more credit card rewards. In some embodiments, the one or more credit card rewards include a purchase refund percentage.

In some embodiments, the one or more steps includes a step to calculate, by the one or more processors, the purchase refund percentage based on the one or more wellness milestones.

In some embodiments, the one or more wellness milestones include a first wellness milestone and a second wellness milestone. In some embodiments, the purchase refund percentage includes a first purchase refund percentage and a second purchase refund percentage. In some embodiments, the one or more steps includes a step to generate, by the one or more processors, the first purchase refund percentage based on the first wellness milestone. In some embodiments, the one or more steps includes a step to generate, by the one or more processors, the second purchase refund percentage based on the second wellness milestone. In some embodiments, the purchase refund percentage includes a cash back percentage. In some embodiments, the cash back percentage includes a purchase amount percentage of one or more consumer products purchased using credit.

In some embodiments, the health monitoring software includes activity tracking software. In some embodiments, the one or more steps includes a step to calculate, by the one or more processors, a user's active motion via the activity tracking software. In some embodiments, the rewards calculation includes an analysis of an active motion of a user. In some embodiments, the second wellness milestone includes a higher active motion amount than the first wellness milestone. In some embodiments, the one or more rewards includes an offer to apply for a credit card.

In some embodiments, the system includes one or more geolocation devices. In some embodiments, the one or more steps includes a step to determine, by the one or more processors, one or more store locations bases on a location of the one or more computers using the one or more geolocation devices. In some embodiments, the one or more financial rewards are associated with the one or more store locations.

In some embodiments, the one or more steps includes a step to generate, by the one or more processors, a mobile wallet via the financial software. In some embodiments, the one or more steps includes a step to link, by the one or more processors, the health monitoring software to the mobile wallet. In some embodiments, the one or more steps includes a step to execute, by the one or more processors, a purchase transaction comprising a transfer of monetary funds from the mobile wallet. In some embodiments, the one or more steps includes a step to calculate, by the one or more processors, a purchase refund percentage based on the one or more wellness milestones.

In some embodiments, the one or more steps includes a step to generate, by the one or more processors, a referral request on a first computer comprising the health monitoring software. In some embodiments, the one or more steps includes a step to send, by the one or more processors, the referral request to a second computer. In some embodiments, the one or more steps includes a step to generate, by the one or more processors, a graphical user interface on the second computer. In some embodiments, the one or more steps includes a step to display, by the one or more processors, an acceptance input configured to enable a second user to execute a referral request acceptance on the second computer. In some embodiments, the one or more steps includes a step to download, by the one or more processors, the health monitoring software on the second computer upon receipt of the acceptance input.

In some embodiments, the one or more steps includes a step to execute, by the one or more computers, a health link between the health monitoring software on the first computer to the health monitoring software on the second computer. In some embodiments, the rewards calculation comprises a first rewards calculation and a second rewards calculation. In some embodiments, the one or more wellness milestones include a first wellness milestone and a second wellness milestone.

In some embodiments, the one or more steps includes a step to the one or more computer readable media further include instructions stored thereon that when executed configure the one or more computers to execute, by the one or more processors, the first rewards calculation on the first computer using the first wellness milestone. In some embodiments, the one or more steps includes a step to execute, by the one or more processors, the second rewards calculation on the second computer using the second wellness milestone. In some embodiments, the first rewards calculation includes the second wellness milestone. In some embodiments, the one or more steps includes a step to the second rewards calculation includes the first wellness milestone.

In some embodiments, the one or more rewards include one or more of changes in life insurance policy benefits, cash back on credit card purchases, exclusive offers from businesses, changes in credit limits, changes in credit interest rates, changes in health insurance rates, changes in auto insurance rates, and discounts on athletic accessories. In some embodiments, the one or more measurements include one or more measurements of exercise, heart rate, blood oxygen level, sleep time, body fat percentage, weight, flexibility, driving habits, food purchases, equipment purchases, and online activity.

### DRAWINGS DESCRIPTION

FIG. 1 depicts a table that lists one or more permutations to prove a rule is correct.

FIG. 2 illustrates a use cases for platform application API requests according to some embodiments.

FIG. 3 shows a customer engagement integration (e.g., Braze™) requester flow diagram according to some embodiments.

FIG. 4 illustrates a Branch SDK plus iOS share sheet workflow according to some embodiments.

FIG. 5 depicts a receiver flow according to some embodiments.

FIG. 6 depicts a system process flow for an offers management module according to some embodiments.

FIG. 7 shows microservice components according to some embodiments.

FIGS. 9-12 illustrate credit card estimations according to some embodiments.

FIG. 13 depicts a non-limiting example of third party platforms integrated into and/or compatible with the present system according to some embodiments.

FIG. 14 illustrates a non-limiting system architecture according to some embodiments.

FIG. 15 illustrates a computer system 910 enabling or comprising the systems and methods in accordance with some embodiments of the system.

### DETAILED DESCRIPTION

In some embodiments, through socialization, the system is configured to enable users to invite others to the platform with a double-sided referral program. In some embodiments, the system is configured to reward multiple parties once a wellness milestone is reached. In some embodiments, the system includes a collaboration and competition system that includes a leaderboard that enables users to compete against other community members using a variety of conventional wearable devices. In some embodiments, the system includes a device-agnostic gamification approach to group wellness tailored to the individual wellness level of each user based upon elevated heart rate minutes.

In some embodiments, the system is configured to enable user to purchase financial products. In some embodiments, the system includes insurance analytics module configured to demonstrate a healthy lifestyle over a longer period of time to insurance underwriters based upon data continuously validated by the system. In some embodiments, the system is configured to offer and issue payment options to users in physical and digital formats using computer (e.g., cloud-based) implemented steps. In some embodiments, the system includes technologies such as credit card as a service (CCaaS) as payment options as a non-limiting example. In some embodiments, the computer implemented steps include a credit card procurement module configured to execute and track the credit card procurement process from application to physical card delivery. In some embodiments, in a situation where a failure occurs, the system includes an orchestration layer configured to provide actionable feedback to address the issue while maintaining the simplicity of the original setup.

In some embodiments, the system is configured to enable users to purchase an item, such as an Apple Watch, on credit and rebate the cost of the product as a reward for achieving pre-determined goals. In some embodiments, the system is configured to send the rebates in time-based installments as a reward for various wellness accomplishments. In some embodiments, the system is configured to send additional reward options include elevated cash back rates based on categorical and merchant-based spending as well as healthy actions such as meditation, annual physical exams, vaccinations, as well as any other conventional health related activity as non-limiting examples according to some embodiments. In some embodiments, the system is configured to calculate a health score and use the health score to determine ongoing membership benefits to associate with one or more of a credit, lifestyle-related financial policies, and insurance policies such as accidental death, disability, health, pet, and other insurance types. In some embodiments, the health score includes one or more wellness milestones.

In some embodiments, as a user continues to build a pattern of wellness history and spending that is regularly rewarded by the system described herein, the system is configured to use information such as heart rate data to confirm activity has occurred rather than the traditional underwriting processes with unvalidated form-based options and a single point-in-time blood pressure and lab readings. In some embodiments, in combination with tailored reward offerings, the system is configured to create a cycle for users to maintain healthy lifestyle habits. In some embodiments, the system is configured to reduce the underlying risk within financial products and to benefit the user with better tailored financial products both from a cost and coverage perspective. For example, through an embedded insurance module, the system is configured to provide a user with a lower-priced life insurance policy by using pertinent data collected by the system. In some embodiments, using collected data reduces user input and insurer decisioning improving upon conventional underwriting techniques. In some embodiments, the system provides a user a path to able to quantify a return on investment for their health.

As a non-limiting example, the system is configured to enable a user to choose a nutrition supplement reward tailored to their health activity. In some embodiments, the system is configured to enable the nutrition supplement company to see depersonalized, yet actionable, additional activities and shopping habits for use in future company decisions according to some embodiments. In some embodiments, the system enables partners to make decisions such as moving from online to offline sales, relying on the collected information to determine which brick and mortar stores users would most likely visit. In some embodiments, the system provides companies with valuable insight to modify marketing strategy to focus on other exercises such as yoga upon realizing yogis were opting into a primarily runner-marketed product.

A non-limiting example of a user journey according to some embodiments is as follows:
1. An existing user tells a new user about the system and provides a referral code generated by the system via a text message according to some embodiments.
2. The new user registers with the system on a Tuesday afternoon, having worked out Monday and Tuesday morning. They link their Apple Watch and credit card account during onboarding using the system interface according to some embodiments.
3. The new user's exercise data for the current week (100 minutes of running with a 50 minute daily max contribution) is retrieved from their wearable by the system and applied towards the weekly goal of 150 elevated heart rate minutes according to some embodiments.
4. The new user reaches 150 elevated heart rate minutes on Wednesday after a 50 minute cycling workout and thus has a one week goal achieving streak.
5. The system offered and enables a user to claim a discount to their local running goods store based on the user's location, top exercise activity, and recent spending on running apparel according to some embodiments.
6. The new user and the existing user both receive a referral reward generated by the platform as a result of the new user hitting a one-week streak wellness milestone or claiming a reward according to some embodiments.
7. In some embodiments, the system is configured to enable a new user to apply for a credit card within minutes via an orchestration layer module that is configured to reduce the time needed for various calls between the platform and the backend systems.
9. In some embodiments, the new user is approved for the credit card by the system and instantly receives a virtual credit card generated and stored by the system.
10. In some embodiments, the system is configured to notify the new user of rewards bonus cash back, encouraging the new user to use the system credit card for various services.
11. In some embodiments, the new user uses their system provided credit card in their mobile wallet to pay for a purchase (e.g. groceries) on the following Monday, earning cash back (e.g. 2.5%).
12. In some embodiments, the new user hits the 150 minute elevated heart-rate goal (i.e., a streak) and receives boosted cashback (e.g 5%). In some embodiments, obtaining a streak results in an additional opportunity to earn and/or select rewards offered by the platform. In some embodiments, the user can attain a wide variety of other goals in addition to or instead of the heart-rate goal. Such other goals can include a streak of days or weeks of activity, certain calorie counts, specific exercise goals, and/or any other health-related goal.
13. In some embodiments, the user receives enhanced financial rewards through tailored financial product offerings such as pet insurance and mortgages provided by the system based upon the user's unified wellness habits (spending, fitness, etc).
14. In some embodiments, the system is configured to offer a new and/or different credit card configuration if a (new/second, third etc.) user presents certain health and wellness milestones. In some embodiments, the health and wellness activity (e.g., four-week streak) and general spending (e.g., more than $1500 per month spend on the Paceline credit card) is used to pre-select and target users to be offered an elite, elevated credit card experience through the platform.
15. In some embodiments, this differentiated, elite credit card provides users with additional rewards not accessible with the original credit card.
16. In some embodiments, the elevated rewards offered with the elite credit card will include items such as 10% cashback deals on select merchants, a life insurance policy, exclusive offers from partners, and more.
17. In some embodiments, the system is configured to distribute financial rewards to (new/second, third etc.) users for achieving health milestones that include maintaining healthy habits across an expanded set of wellness dimensions such as sleep, diet, and mindfulness.

In some embodiments, the system is configured to record and/or display a completion streak of multiple wellness activities (e.g., repetitively meeting meditation, exercise minutes, any conventional wellness goal, and/or combinations of each) within a timeframe. In some embodiments, the system is configured to display and/or issue a reward for a completion streak according to some embodiments described herein. In some embodiments, since invites are anonymous when created, there will be situations when invites are created for the same invitee-inviter pair. For instance, two friends send each other invites at the same time. Another scenario supported by system computer implemented instructions: an inviter keeps inviting a reluctant friend who keeps denying the invite. In some embodiments, the system is configured to automatically handle duplicates in multiple ways. In some embodiments, the system is configured to implement an async “processing” lambda that removes old duplicates and/or consolidates them. In some embodiments, the system is configured to implement an abusive user discovery program when a problem with users abusing the 1:1 sharing feature is detected. In some embodiments, the system is configured to prevent more than one invite between inviter and invitee by querying for at least one invite where status=accepted.

In some embodiments, when finding duplicates (same inviter and invitee), the system is configured to remove all of the duplicates except the one that satisfies the following criteria: always choose the newer invite unless the newer one is received and older one is accepted. In some embodiments, this rule actually helps prevent two users from having more than one accepted invite. In some embodiments, “newer” refers to the date the invite was accepted, not the date it was created.

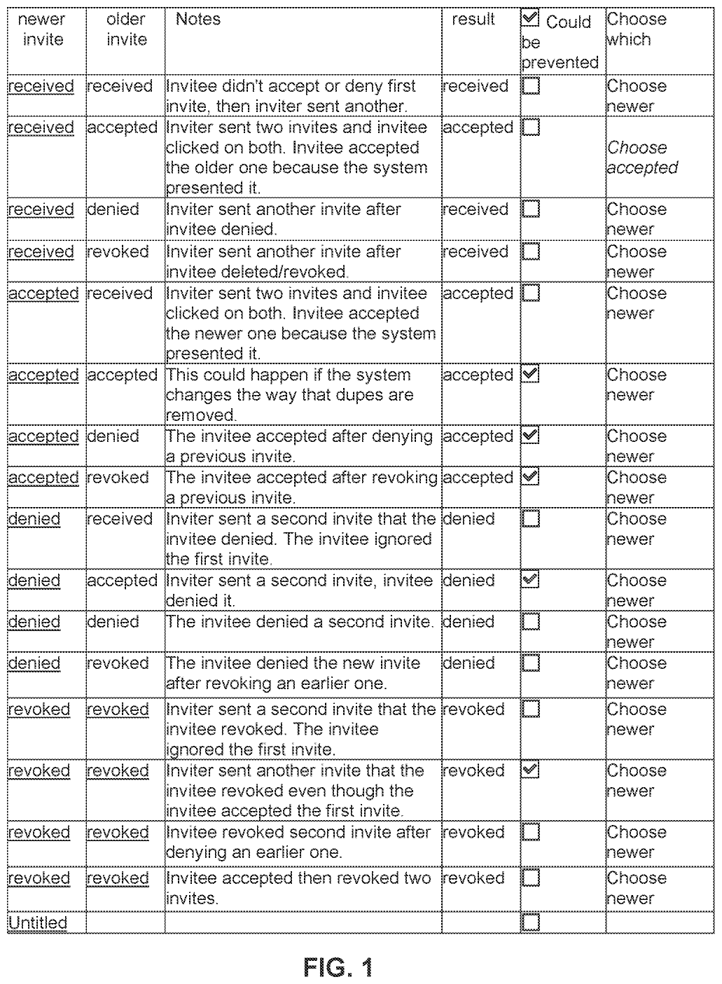

FIG. 1 depicts a table that lists all the possible permutations to prove a rule is correct. In some embodiments, some permutations should actually be impossible (prevented) because of this rule (See checkboxes), but because, at scale, things that seem impossible become possible, the system is configured to make sure all possibilities are able to be handled.

In some embodiments, instructions stored on one or more non-transitory computer readable media when executed cause the one or more computers to:
request, by one or more processors, one or more user invites via a query;
partition, by the one or more processors, the one or more invites into the following groups:
   a. “new” invites that have no invitee (these can simply be converted to friend “stubs” by the one or more processors according to some embodiments).
   b. “accepted” invites that should be paired with user summary and statistical data (name, progress, etc.);
   c. “other” invites (e.g., “received”, “revoked”) that should be paired with only summary data;
request and/or receive, by the one or more processors, all the user summary and statistical data;
create, by the one or more processors, complete friend entities;

request and/or receive, by the one or more processors, the user summary data; and

create, by the one or more processors, friend entities with no stat data (just names);

concatenate, by the one or more processors, the results of the queries for all of the above and return in the HTTP response.

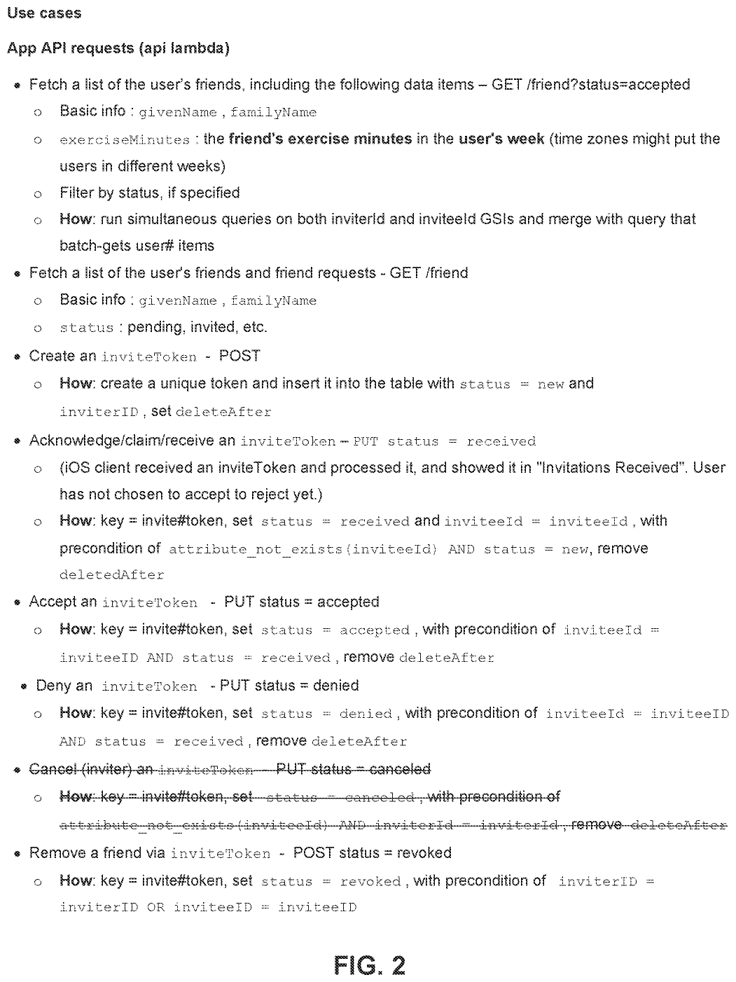

FIG. 2 illustrates use cases for platform application API requests according to some embodiments.

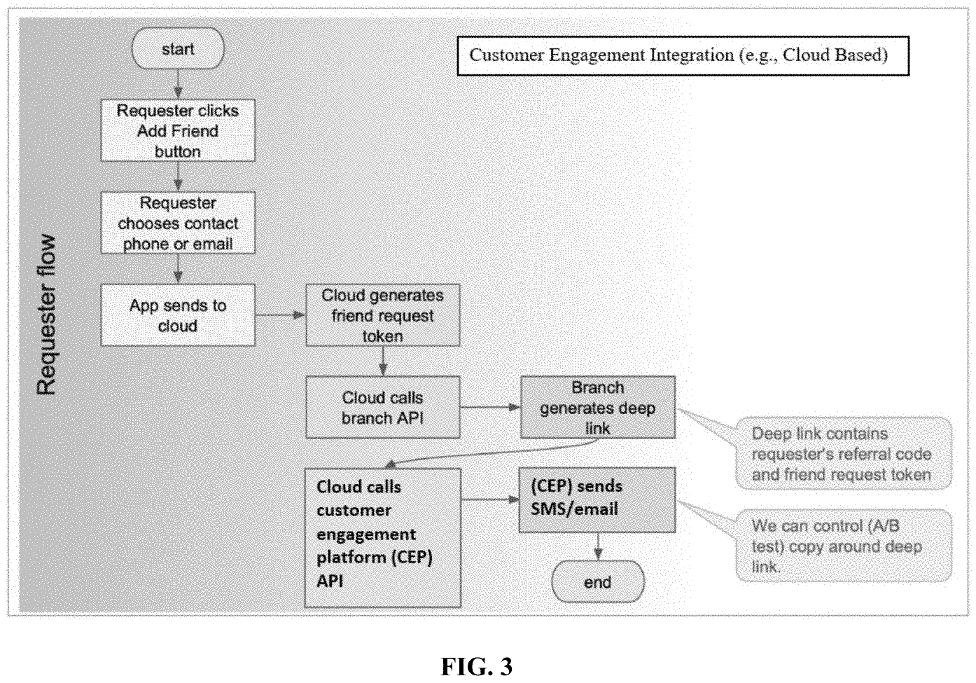

In some embodiments, the requesting user (inviter) is presented with the contacts list, chooses a contact (receiver) by selecting the contact's phone or email, and the app (any reference to an app is also a reference to the platform) sends this info to the system, at least a portion of which may be located in a cloud server (a reference to a cloud server is also a reference to a general server). In some embodiments, the cloud calls branch to create a deep link and then sends this info to the customer engagement platform. In some embodiments, the customer engagement platform may include Braze™ as a non-limiting example. In some embodiments, Braze™ sends an SMS or email message to the receiver after testing a copy. FIG. 3 shows a Braze™ requester flow diagram according to some embodiments.

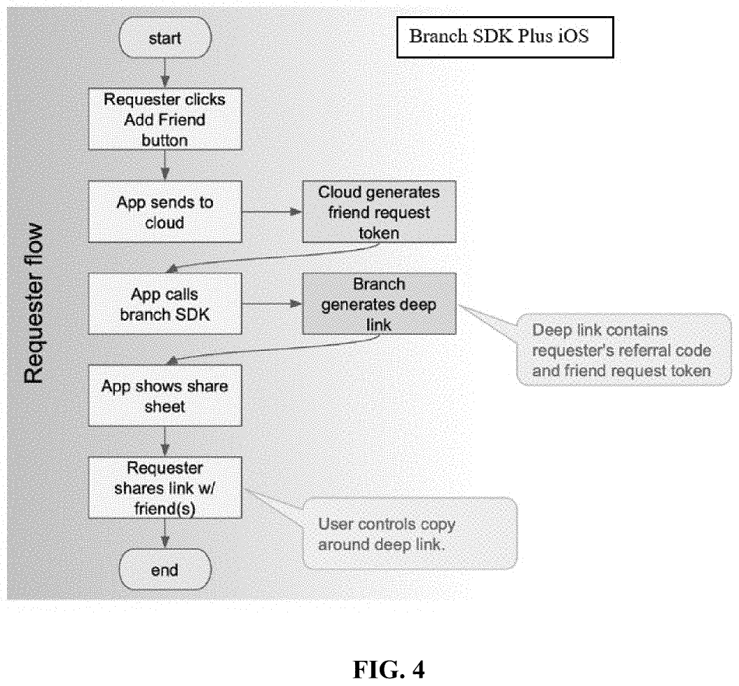

In some embodiments, the system is configured to implement a branch SDK plus iOS share sheet according to some embodiments. In some embodiments, the requesting user (inviter) is presented with the standard iOS share sheet and follows the familiar iOS sharing flow. In some embodiments, behind the scenes before presenting the share sheet, the app calls the platform and the branch SDK to create a deep link. In some embodiments, this system functionality provides the following benefits: Invitation comes directly from a friend; user can share via SMS and email, as well as WhatsApp, iMessage, Instagram, Twitter, Facebook, etc.; more familiar, less intrusive user flow; less cloud work; less overall complexity; no need to use the Branch HTTP API (Branch SDK is already in the app); the system already uses the share sheet. FIG. 4 illustrates a branch SDK plus iOS share sheet workflow according to some embodiments.

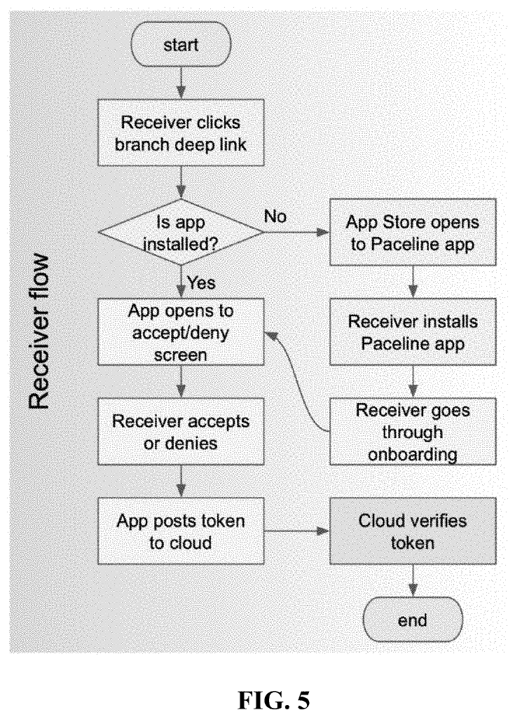

In some embodiments, the receiving user (invitee) receives a friend request sent by the system via SMS or email (or other form of communication, such as iMessage, Instagram, Facebook, Twitter, etc., depending on the Inviter flow chosen). In some embodiments, when the receiver taps the deep link in the friend request via a system provided interface, one of two sub-flows happens. If the system app is already installed, the app is configured to open directly to the Accept/Deny screen where the user can accept or deny the friend request. In some embodiments, the system is configured to verify the friend request. In some embodiments, if the user does not already have the system app installed, the system is configured to direct the user to the App Store to install it. In some embodiments, after successfully installing the system app and going through the onboarding flow, the user follows the flow described above. FIG. 5 depicts a receiver flow according to some embodiments.

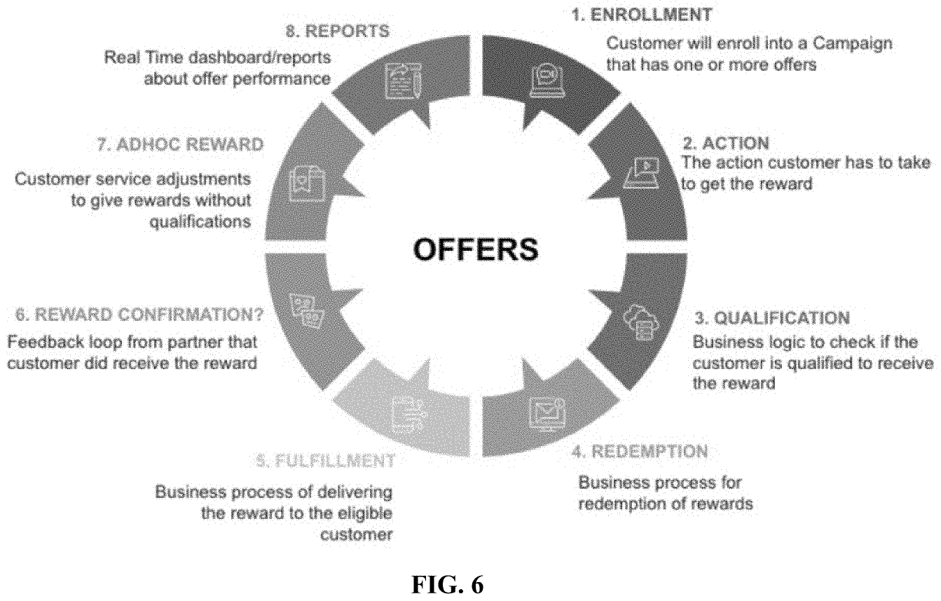

FIG. 6 depicts a system process flow for an offers management module according to some embodiments. In some embodiments, of the entire offers management system, the qualification microservice handles the Enrollment, Action, and Qualification pieces as shown in FIG. 6. In some embodiments, the Redemption and Fulfillment pieces would be satisfied by downstream services such as a reward microservice or a railsbank-integration microservice, as non-limiting examples.

In some embodiments, the system is configured to implement a boost cash back program. In some embodiments, the following terms are defined by their computer implemented step:
Action: an event that should be logged for a user. In some embodiments, actions are typically only logged if the affected user is enrolled. In some embodiments, actions are typically events inferred from messages received from other microservices.
Enroll: to start watching and recording Actions for a specific user
Enrollment: an event that signals the system to Enroll a user
Unenroll: to stop watching and recording Actions for a specific user.
Qualify: to detect that a predefined set of Actions has been completed and logged.
Qualification: an event declaring that a user has completed the set of Actions, published for consumption by other microservices according to some embodiments.
Redeem: to perform the necessary steps to ensure the offer will be fulfilled.
Redemption: an event announcing that the user has redeemed an offer.
Offer: a collection of related Enroll, Actions, Qualify, etc. concepts with a specific objective and time period.

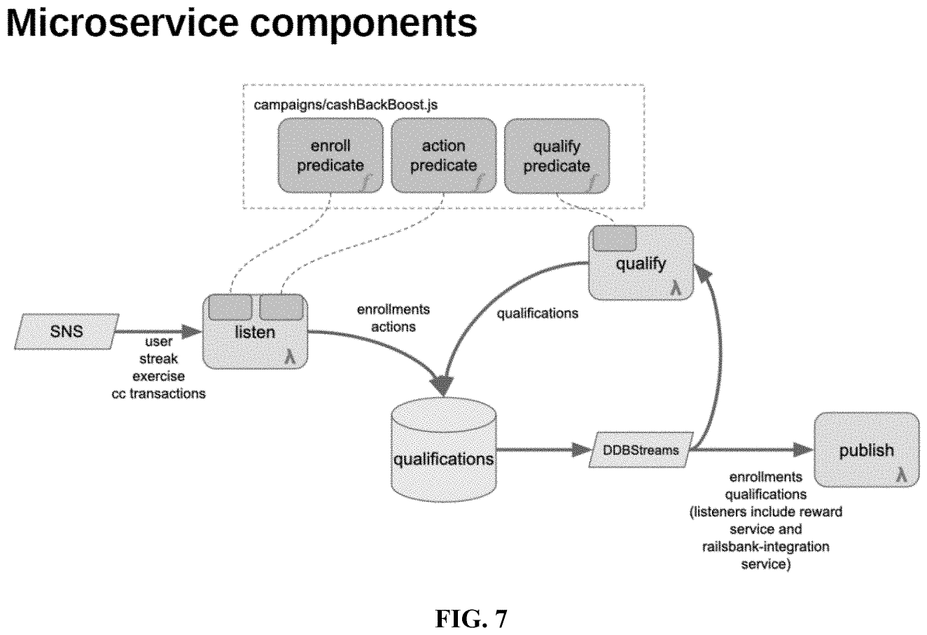

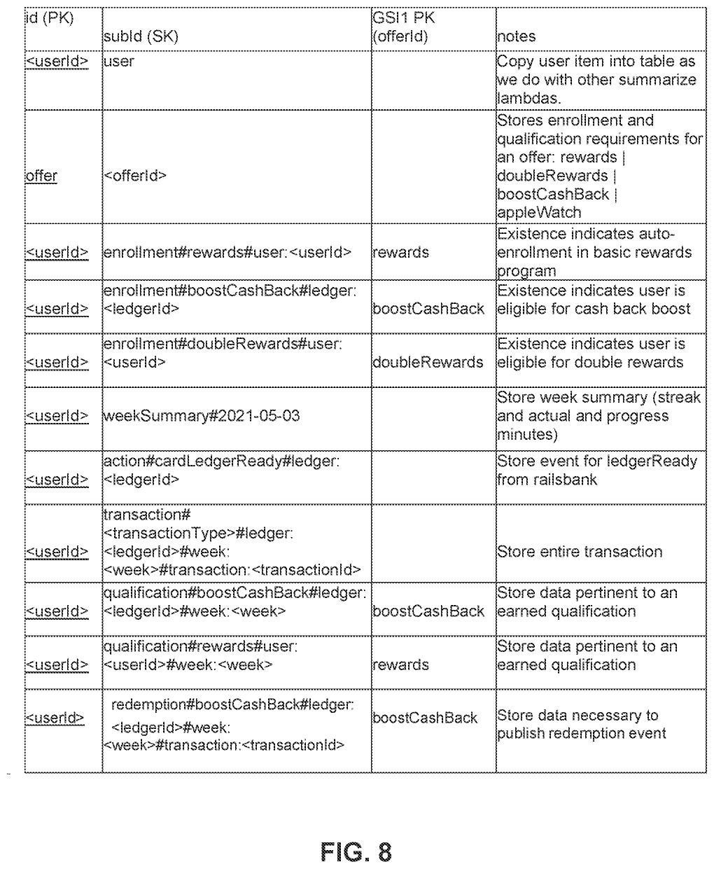

FIG. 7 shows microservice components according to some embodiments. In some embodiments, a microservice includes a collection of lambdas (“listeners” instruction execution) that are configured to listen to various messages from other parts of the system. In some embodiments, these messages are filtered, formatted, and (if pertinent) created and logged in the table as Actions, Summaries, Enrollments, or Unenrollments. In some embodiments, a lambda (“qualify” execution) watches for Actions and determines if the user has completed all actions to qualify. If so, a Qualification item is created and stored in the table according to some embodiments. A lambda (“publish” execution) watches for new Redemption items and publishes them so that other portions of the system can start the fulfillment process.

In some embodiments, an “offer” execution contains computer implemented steps for enrollment as a list of expected record types and requirements for qualification as a map of predefined parameters. In some embodiments, Enrollment steps and Qualification requirements are copied into “enrollment” records. In some embodiments, these are used to determine if incoming actions or summary updates should trigger enrollment or qualification. In some embodiments, “listeners” execution transforms incoming messages into action or summary object and stores in local db. In some embodiments, “ingest” execution listens to dynamodb streams and searches for offers requiring the existence of incoming records for enrollment. If found, and a corresponding “enrollment” status is not found for a user, an “enrollment” status is created. In some embodiments, pending enrollments for users are checked as well, and if there is an enrollment Step for the incoming record, that list is updated and, if full, the enrollment status is changed to “active” status.

In some embodiments, “qualify” execution listens for actions and summary updates in dynamodb streams and compares items against user enrollment qualification requirements. In some embodiments, if any enrollments are found that require the incoming item, all requirements are compared against actions and summaries currently stored. If all requirements are satisfied a “qualification” record is stored.

In some embodiments, an incoming user mutation event (create or update) is stored in the table as both a “user” and as an enrollment into the basic rewards program (e.g. “enrollment #rewards”). In some embodiments, an incoming credit card event that indicates card is activated is stored in the table (e.g. “action #cardActivated”). In some embodiments, enroll predicate would then add enrollment records the database (e.g. “enrollment #cashBackBoost” and “enrollment #doubleRewards”). In some embodiments, an incoming credit card transaction event is stored (e.g. “transaction #<transactionId>”).

In some embodiments, the qualify lambda picks up transaction insert, fetches enrollments that include transaction in “qualificationTriggers” field (ie: “enrollment #cashBackBoost”) and if items exist, compares qualifiers field in enrollment row with data present for user. (ie: “streaks” calculated from “week #?” items). If exerciseMinutes>150 (streakAchieved===true?), stores “qualification #cashBackBoost #<transactionId>” item for every cash back transaction type during that week. In some embodiments, each new “enrollment #?” and “qualification #?” insert or delete DynamoDBStreams event is published as a mutation event. FIG. 8 shows a table of qualification data types, Ids, and SubIds according to some embodiments.

In some embodiments, the system includes data operations. In some embodiments, “rules” include: all writes (putItem, updateItem) need to know the exact value of the PK and SK on the table; all queries need to know either the exact value of the PK on the table or a GSI's PK, knowing the SK or a prefix for the SK is optimal; and all direct gets (getItem) need to know the exact value of the PK and SK on the table.

In some embodiments, read (query, getItem) include the following cases:
1. Get all (query) enrollment triggers when an item is inserted. E.g.:
   PK: id=“offer”;
   Filter (in code): enrollmentTriggers matches an action (ex:
   action.subId.startsWith(offer.enrollmentTrigger))
2. Get all (query) enrollment items for a user and offer. E.g.:
   PK: id=userId;
   SK: begins_with(subId, “enrollment #”)
   Filter (in code): any qualificationTriggers value matches an action (ex:
   enrollment.qualification Triggers.forEach(t⇒action.subId.startsWith(t))
3. Get all (query) qualification items for a user and offer. E.g.:
   PK: id=userId;
   SK: begins_with(subId, “qualification #”)
   Filter (in code): any redemptionTriggers value matches an action (ex:
   qualification.redemptionTriggers.forEach(t⇒action.subId.startsWith(t))
4. [Optional] Get all enrollment or qualification items for an offer so they can be updated. E.g.:
   GSI1 PK: offerId=X
   In some embodiments, Write (putItem) cases include:
1. Put week summary, user, transaction. E.g.:
   PK: id=userId
   SK: (varies)
   Metadata: createdAt (TTL), eventDate(?)
   Qualification microservice 8
2. Put enrollment. E.g.:
   PK: id=userId
   SK: subId=“enrollment #<offerId>”
   Metadata: offend (GSI1 PK), createdAt (TTL), eventDate(?)
   Precondition: “attribute_not_exist(subId)”
3. Put qualification. E.g.:
   PK: id=userId
   SK: subId=“qualification #<enrollmentId>#<periodId>”
   Metadata: offerId (GSI1 PK), createdAt (TTL), eventDate(?)
   Precondition: “attribute_not_exist(subId)”
4. Put redemption. E.g.:
   PK: id=userId
   SK: subId=“redemption #<qualificationId>[#optionalReferenceId]”
   Metadata: offerId (GSI1 PK), createdAt (TTL), eventDate(?)
   Precondition: “attribute_not_exist(subId)”

In some embodiments, the system includes a security module. In some embodiments, industry best practices indicate one should protect financial data behind more secure protocols than the rest of the system's data. In some embodiments, the simplest solution would be to change the security protocols of the entire system, but this would degrade the user experience for the non-financial parts because the more secure protocol is more burdensome. In some embodiments, a better solution is to have two levels of security: one for the financial data and one for other data (including the background HealthKit process for users that have selected Apple Watch as their wellness device). In some embodiments, the following description outlines details of the system that provide an additional, more secure protocol, a.k.a. L2 Security. In some embodiments, the app uses biometrics (FaceID or TouchID) to ease the authentication process for the user.

A useful way to talk about security levels is as if they are security gates. The user must cross through gates to access restricted areas, passing through deeper and deeper gates to access more and more restricted data. This concept is useful when discussing the app screens, but hides one important detail: it's the data, not the application screens, that determine the boundaries of the gates. In short, if we mix data from differing security levels into a single screen, the gate concept becomes murky.

In some embodiments, a more precise concept is security levels on the APIs. In some embodiments, the system includes API endpoints that fall into at least two levels In some embodiments, those levels include: L0: No authentication required (e.g. login, configuration); and L1: Authentication required. In some embodiments, the financial features of our app require a new, more secure level: L2.

In some embodiments, the system includes security protocols that requires a username (email) and a strong password for authentication. In some embodiments, in exchange for a correct username-password pair, the authentication service (auth microservice) returns a set of tokens: a session token, a refresh token, and an access token. In some embodiments, the session token is short-lived and contains the identity and the capabilities of the user. In some embodiments, the access token is short-lived and can be used to modify a user's authentication information (username and password). In some embodiments, the refresh token is long-lived token and can be used to obtain new session and access tokens. In some embodiments, once a refresh token expires, the app prompts the user to re-authenticate to get a whole new set of tokens. The current short-lived tokens are valid for 5 minutes. In some embodiments, the long-lived refresh token is valid for 1 year (but may be as long as 10 years or longer, depending on the systems and/or partner policies which are applicable.)

In some embodiments, the app requests its initial tokens (a.k.a. authenticates) using a “POST/auth/session” endpoint. In some embodiments, the lifetime of the session and access tokens is 5 minutes. In some embodiments, the lifetime of the refresh token is 1 year (10 years is desired). In some embodiments, when a token is no longer valid, the API endpoints return a 401 status code to the app. In some embodiments, when the app receives a 401 status code from the API endpoints, it requests a new set of tokens from the “POST/auth/session” endpoint. In some embodiments, if the call to this endpoint returns a 4xx status code, the app drops its tokens and starts the authentication process over.

In some embodiments, the app operates on the L0 and L1 API endpoints. In some embodiments, a change is that the refresh tokens will last 10 years, not 1 year. In some embodiments, the lifetime of the tokens that authenticate the L2 endpoints are no more than 15 minutes of inactivity. In some embodiments, the tokens may last longer if the user is actively using the application. In some embodiments, the tokens are limited to some reasonably pre-determined period of time. In some embodiments, this is achieved by limiting the lifetime of the refresh tokens for these endpoints to the minimum (e.g., 60 minutes) and implementing custom logic to detect inactivity.

In some embodiments, acceptance criteria include the following:
1. In some embodiments, when the app accesses an L1 API endpoint, it behaves as configured.
2. In some embodiments, when the app accesses an L2 API endpoint, the endpoint will reject the request with a 401 status code if the app has not sent a valid short-term session token along with the request. (Conversely, if the app sends a valid short-term session token, the endpoint will return a 2xx status code, assuming there are no other errors.)
3. In some embodiments, when the app receives a 401 status code from an L2 endpoint (i.e. the short-lived tokens expire), it will request a new short-lived token from a new L2 auth endpoint (such as POST/authL2/session).
4. In some embodiments, when the app receives a 4xx status code from the L2 auth endpoint, it will start the authentication process against the new L2 auth endpoint by prompting the user for L2 Security 4 their username and password.
5. In some embodiments, the user can authenticate against the new L2 auth endpoint using the same username and password pair that they use for the L1 auth endpoint.
6. In some embodiments, when the app accesses any L2 endpoint after a period of time (TBD), the L2 endpoint will return a 401 status code.

In some embodiments, a cloud API is split into two APIs: the existing L0/L1 API defined in openapi.json and a new L2 API. In some embodiments, the app's network layer is split into two classes, but they share the same authentication class. In some embodiments, the app introduces a new authentication class that will handle the calls to the L2 API.

In some embodiments, The outcome is a new L2 API at a url. In some embodiments, all of the following endpoints are located at the L2 API: /credit/*. In some embodiments, a new url under the L1 API, authenticates the user's credentials (or refresh the tokens) and return the short-lived tokens to be used against the L2 API. In some embodiments, a Cognito pre-authentication trigger lambda injects inactivity timeout claims into (one of) the JWT tokens. In some embodiments, each endpoint handler checks this inactivity timeout claim to determine if the inactivity timeout has expired. If so, in some embodiments, the refresh token and/or all tokens are revoked via the revokeToken Cognito API and the endpoint will return a 401 status code.

In some embodiments, the system includes a Cognito App with 60 min refresh tokens in core-services/sam.json. In some embodiments, the system includes an L2 API Gateway in app-api/sam.json (or a microservice). In some embodiments, the system includes a L2 openapi.json in app-api (or a microservice). In some embodiments, the system includes Copy /credit/* endpoints from L1 openapi.json to new L2 openapi.json. In some embodiments, the system includes an L2 auth endpoint to existing L1 openapi.json (e.g. a′POST/auth/l2session″). In some embodiments, the system includes an L2 auth http handler (using new Cognito App) to auth service. In some embodiments, the system includes a handler to all L2 endpoints that checks the inactivity timeout JWT claim. In some embodiments, logout endpoint revokes as many tokens as possible.

In some embodiments, the system includes a Cognito App with 60 min refresh tokens in core-services/sam.json. In some embodiments, the system includes a Cognito App to existing openapi.json. In some embodiments, the system includes an L2 auth endpoint to existing openapi.json (e.g. POST/auth/l2session). In some embodiments, the system includes an L2 auth http handler (using new Cognito App) to auth service. In some embodiments, the system includes an L2 Security 6. In some embodiments, the system includes a handler to all L2 endpoints that checks the inactivity timeout JWT claim. In some embodiments, the system includes a create new Cognito trigger lambda that injects inactivity timeout into JWT claims. In some embodiments, the system is configured to segment network layer by path (/credit/* endpoints vs all others) and ensure token refresh process and credentials prompts work for both L1 and L2 network layers.

Referral codes are implemented generically as access codes (“accessCode”). In some embodiments, access codes are codes that the users enter while onboarding and may be used for several purposes. In some embodiments, a first use case for access codes is to prevent too many users from creating accounts. In some embodiments, a second use case is to use these codes to track referrals. In some embodiments, each user is assigned a unique access code (called “referralCode” in the “users” table). In some embodiments, the system is configured to enable users to give this code to other users to use during onboarding. In some embodiments, by recording every user's referral code and access code, the system is configured to construct a social graph.

In some embodiments, the low-level features of the original access-codes microservice include:
1. Limit a code by the number of claims by new users (claimCount vs claimLimit) according to some embodiments.
2. Limit a code by date range (startDate≤x<expireDate) according to some embodiments.
3. Limit an access code to a one-time usage (not isReusable) according to some embodiments.
4. Associate a code with a promotion (promotionCode). In some embodiments, referral codes use “referral” as the promotion code.

In some embodiments, if an API endpoint is exposed to the app that allows users to request new access codes, this endpoint could be used by a malicious user to create hundreds, thousands, or even millions of access codes. In some embodiments, this causes several problems, such as blocking most users from being able to complete onboarding. In some embodiments, by restricting the functionality of this endpoint, we may be able to mitigate many forms of attack. For instance, if we validate that the requested new code is a variation of the user's existing code, the user would not be able to affect other users according to some embodiments. In some embodiments, the number of unique codes is approximately 887 million. In some embodiments, since these codes are assigned using a hashing algorithm, the chances of two users getting the same code is 1/887,503,681.

In some embodiments, the system is configured to Associate all referral codes with users in the database (“ownerId?: string”). In some embodiments, when creating a new referral code, enforce a rule that ensures it's a variation of the requesting user's referral code. In some embodiments, the system is configured to look up a users's referral codes by “ownerId” and compare (GSI). In some embodiments, the system is configured to associate user-generated referral codes with a marketing category (“category: string”). In some embodiments, the system is configured to associate all access codes with users in the database (“claimedByIds?: string[ ]”).

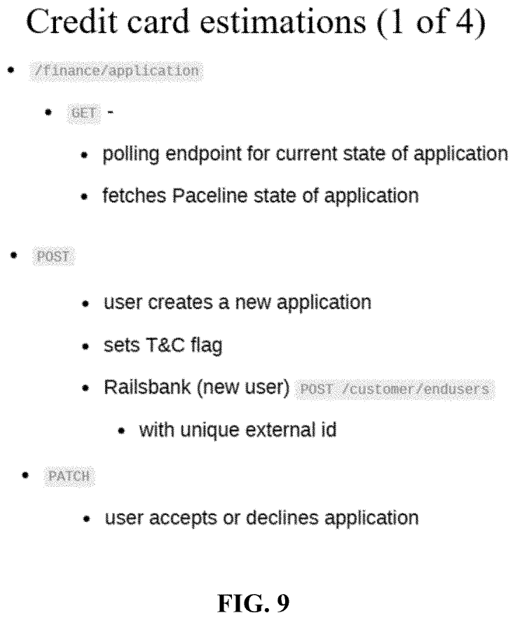

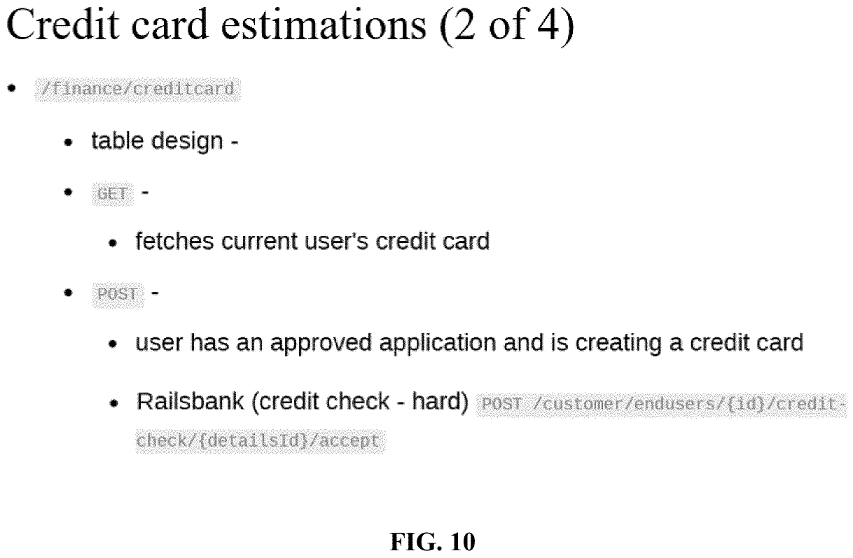

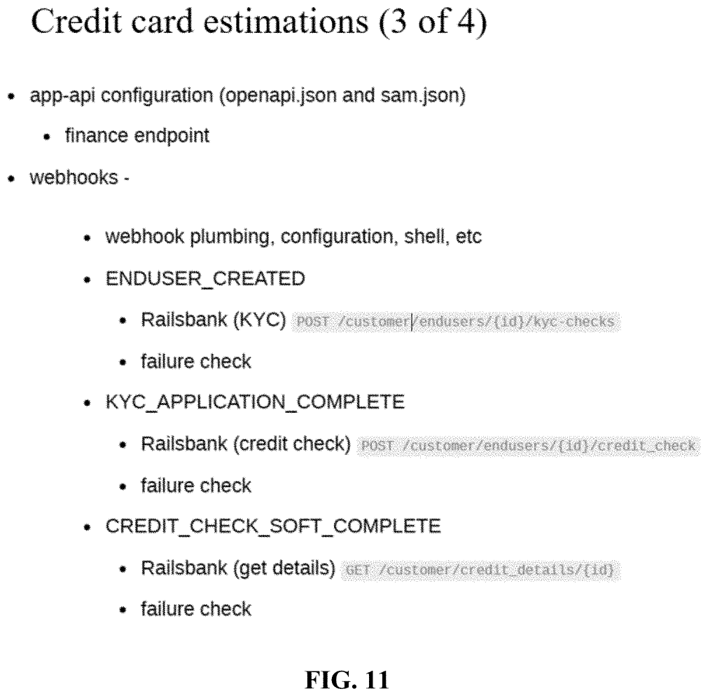

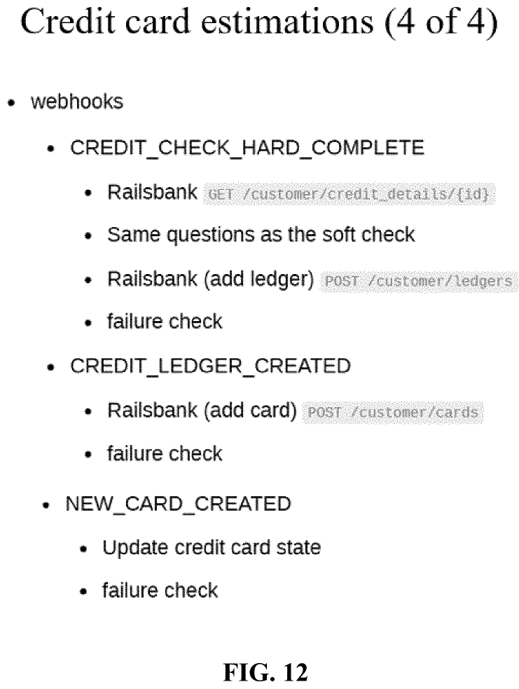

FIGS. 9-12 illustrate credit card estimations according to some embodiments.

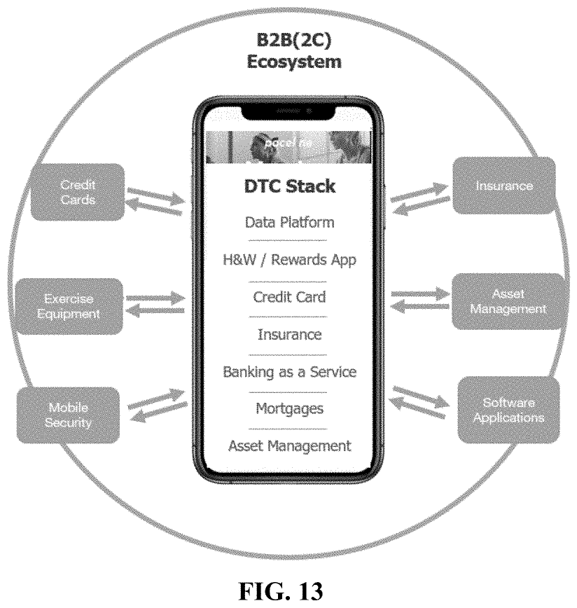

FIG. 13 depicts a non-limiting example of third party platforms integrated into and/or compatible with the present system according to some embodiments. In some embodiments, the system includes a unified wellness platform that is designed to incentivize and strengthen physical, mental, and financial health together, in a continuous virtuous cycle and personalized to each participant. In some embodiments, the system includes a mobile-first, Direct to Consumer (DTC) approach configured to drive a community of highly engaged and passionate users who will grow to recognize the true value of their health and participate in the economics of it. In some embodiments, the system includes a powerful Business to Business (B2B) ecosystem configured to enable embedded financial services as well as other partner platforms. In some embodiments, the system is configured to accept payment from third party platforms.

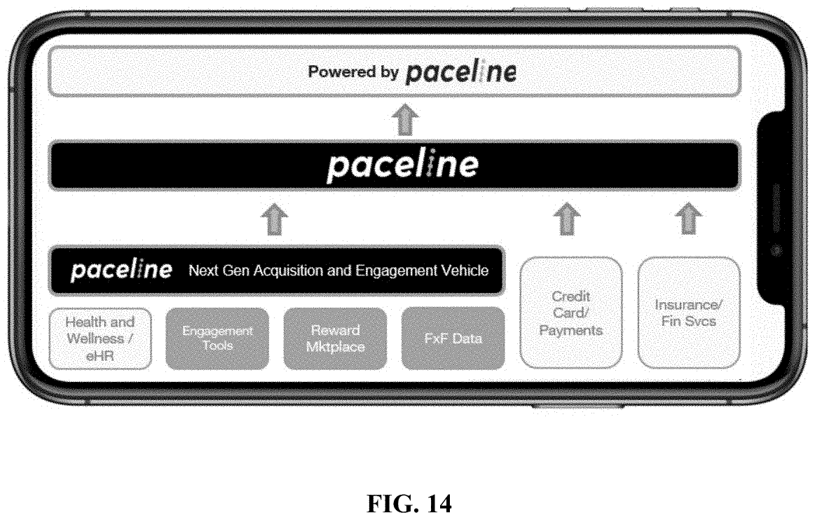

FIG. 14 illustrates a non-limiting system architecture according to some embodiments. In some embodiments, the system is configured to enable integration of modern architecture that customized in a variety of expressions to drive next gen engagement through any/every channel such as DTC and B2B2C (Business to Business to Consumer) as non-limiting examples. In some embodiments, the system is configured to be paired with an OpenAPI ecosystem. In some embodiments, the system includes foundational pillars guiding how the system delivers the most complete and engaging offering to customers. In some embodiments, the system is configured to enable flexibility to support co-creation and partnership opportunities to supplement strengths.

In some embodiments, the system architecture includes one or more of integrated electronic health records (eHR), engagement tools, a rewards marketplace(s), Fitness and Finance (FxF) (Fitness+Financial), credit card payment platform(s), insurance platforms, and financial services platforms. In some embodiments, the system is configured to turn fitness into a financial currency. In some embodiments, the system is configured to enable integration of the system into third party platforms. In some embodiments, the system is configured to enable expansion and/or addition of health and wellness (H&W) categories.

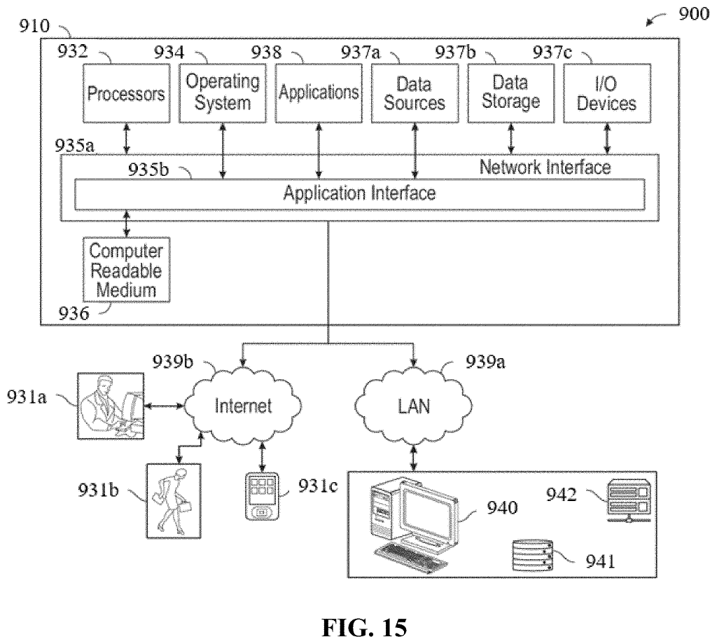

FIG. 15 illustrates a computer system 910 enabling or comprising the systems and methods in accordance with some embodiments of the system. In some embodiments, the computer system 910 can operate and/or process computer-executable code of one or more software modules of the aforementioned system and method. Further, in some embodiments, the computer system 910 can operate and/or display information within one or more graphical user interfaces (e.g., HMIs) integrated with or coupled to the system.

In some embodiments, the computer system 910 can comprise at least one processor 932. In some embodiments, the at least one processor 932 can reside in, or coupled to, one or more conventional server platforms (not shown). In some embodiments, the computer system 910 can include a network interface 935a and an application interface 935b coupled to the least one processor 932 capable of processing at least one operating system 934. Further, in some embodiments, the interfaces 935a, 935b coupled to at least one processor 932 can be configured to process one or more of the software modules (e.g., such as enterprise applications 938). In some embodiments, the software application modules 938 can include server-based software and can operate to host at least one user account and/or at least one client account, and operate to transfer data between one or more of these accounts using the at least one processor 932.

With the above embodiments in mind, it is understood that the system can employ various computer-implemented operations involving data stored in computer systems. Moreover, the above-described databases and models described throughout this disclosure can store analytical models and other data on computer-readable storage media within the computer system 910 and on computer-readable storage media coupled to the computer system 910 according to various embodiments. In addition, in some embodiments, the above-described applications of the system can be stored on computer-readable storage media within the computer system 910 and on computer-readable storage media coupled to the computer system 910. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 910 can comprise at least one computer readable medium 936 coupled to at least one of at least one data source 937a, at least one data storage 937b, and/or at least one input/output 937c. In some embodiments, the computer system 910 can be embodied as computer readable code on a computer readable medium 936. In some embodiments, the computer readable medium 936 can be any data storage that can store data, which can thereafter be read by a computer (such as computer 940). In some embodiments, the computer readable medium 936 can be any physical or material medium that can be used to tangibly store the desired information or data or instructions and which can be accessed by a computer 940 or processor 932. In some embodiments, the computer readable medium 936 can include hard drives, network attached storage (NAS), read-only memory, random-access memory, FLASH based memory, CD-ROMs, CD-Rs, CD-RWs, DVDs, magnetic tapes, other optical and non-optical data storage. In some embodiments, various other forms of computer-readable media 936 can transmit or carry instructions to a remote computer 940 and/or at least one user 931, including a router, private or public network, or other transmission or channel, both wired and wireless. In some embodiments, the software application modules 938 can be configured to send and receive data from a database (e.g., from a computer readable medium 936 including data sources 937a and data storage 937b that can comprise a database), and data can be received by the software application modules 938 from at least one other source. In some embodiments, at least one of the software application modules 938 can be configured within the computer system 910 to output data to at least one user 931 via at least one graphical user interface rendered on at least one digital display.

In some embodiments, the computer readable medium 936 can be distributed over a conventional computer network via the network interface 935a where the system embodied by the computer readable code can be stored and executed in a distributed fashion. For example, in some embodiments, one or more components of the computer system 910 can be coupled to send and/or receive data through a local area network (“LAN”) 939a and/or an internet coupled network 939b (e.g., such as a wireless internet). In some embodiments, the networks 939a, 939b can include wide area networks (“WAN”), direct connections (e.g., through a universal serial bus port), or other forms of computer-readable media 936, or any combination thereof.

In some embodiments, components of the networks 939a, 939b can include any number of personal computers 940 which include for example desktop computers, and/or laptop computers, or any fixed, generally non-mobile internet appliances coupled through the LAN 939a. For example, some embodiments include one or more of personal computers 940, databases 941, and/or servers 942 coupled through the LAN 939a that can be configured for any type of user including an administrator. Some embodiments can include one or more personal computers 940 coupled through network 939b. In some embodiments, one or more components of the computer system 910 can be coupled to send or receive data through an internet network (e.g., such as network 939b). For example, some embodiments include at least one user 931a, 931b, is coupled wirelessly and accessing one or more software modules of the system including at least one enterprise application 938 via an input and output (“I/O”) 937c. In some embodiments, the computer system 910 can enable at least one user 931a, 931b, to be coupled to access enterprise applications 938 via an I/O 937c through LAN 939a. In some embodiments, the user 931 can comprise a user 931a coupled to the computer system 910 using a desktop computer, and/or laptop computers, or any fixed, generally non-mobile internet appliances coupled through the internet 939b. In some embodiments, the user can comprise a mobile user 931b coupled to the computer system 910. In some embodiments, the user 931b can connect using any mobile computing 931c to wireless coupled to the computer system 910, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of health and wellness by integrating of financial rewards into improved health activity tracking systems. The disclosure describes the specifics of how a machine including one or more computers comprising one or more processors and one or more non-transitory computer implement the system and its improvements over the prior art. The instructions executed by the machine cannot be performed in the human mind or derived by a human using a pin and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide a technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless a explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system including:
health monitoring software, and
one or more computers comprising one or more processors and one or more computer readable media, the one or more computer readable media including instructions stored thereon that when executed configure the one or more computers to:
receive, by the one or more processors, a request for a link to the health monitoring software;
receive, by the one or more processors, one or more measurements from one or more sensors electronically coupled to the health monitoring software;
compare, by the one or more processors, the one or more measurements to one or more wellness milestones;
execute, by the one or more processors, a rewards calculation comprising one or more rewards associated the one or more wellness milestones; and
send, by the one or more processors, the one or more rewards to the health monitoring software.

2. The system of claim 1,
wherein the health monitoring software comprises financial software; and
wherein the one or more rewards include one or more financial rewards.

3. The system of claim 2,
wherein the one or more financial rewards include one or more credit card rewards.

4. The system of claim 3,
wherein the one or more credit card rewards include a purchase refund percentage; and
wherein the one or more computer readable media include further instructions stored thereon that when executed configure the one or more computers to:
calculate, by the one or more processors, the purchase refund percentage based on the one or more wellness milestones.

5. The system of claim 4,
wherein the one or more wellness milestones include a first wellness milestone and a second wellness milestone;
wherein the purchase refund percentage includes a first purchase refund percentage and a second purchase refund percentage;
wherein the one or more computer readable media include further instructions stored thereon that when executed configure the one or more computers to:
generate, by the one or more processors, the first purchase refund percentage based on the first wellness milestone; and
generate, by the one or more processors, the second purchase refund percentage based on the second wellness milestone.

6. The system of claim 5,
wherein the purchase refund percentage includes a cash back percentage.

7. The system of claim 6,
wherein the cash back percentage includes a purchase amount percentage of one or more consumer products purchased using credit.

8. The system of claim 7,
wherein the health monitoring software includes activity tracking software;
wherein the one or more computer readable media include further instructions stored thereon that when executed configure the one or more computers to:
calculate, by the one or more processors, a user's active motion via the activity tracking software;
wherein the rewards calculation includes an analysis of an active motion of a user; and
wherein the second wellness milestone includes a higher active motion amount than the first wellness milestone.

9. The system of claim 3,
wherein the one or more rewards includes an offer to apply for a credit card.

10. The system of claim 2, further comprising:
one or more geolocation devices;
wherein the one or more computer readable media further include instructions stored thereon that when executed configure the one or more computers to:
determine, by the one or more processors, one or more store locations bases on a location of the one or more computers using the one or more geolocation devices;
wherein the one or more financial rewards are associated with the one or more store locations.

11. The system of claim 2, further comprising:
wherein the one or more computer readable media further include instructions stored thereon that when executed configure the one or more computers to:
generate, by the one or more processors, a mobile wallet via the financial software;
link, by the one or more processors, the health monitoring software to the mobile wallet;
execute, by the one or more processors, a purchase transaction comprising a transfer of monetary funds from the mobile wallet;
calculate, by the one or more processors, a purchase refund percentage based on the one or more wellness milestones.

12. The system of claim 11, further comprising:
wherein the one or more computer readable media further include instructions stored thereon that when executed configure the one or more computers to:
generate, by the one or more processors, a referral request on a first computer comprising the health monitoring software;
send, by the one or more processors, the referral request to a second computer;
generate, by the one or more processors, a graphical user interface on the second computer;
display, by the one or more processors, an acceptance input configured to enable a second user to execute a referral request acceptance on the second computer; and
download, by the one or more processors, the health monitoring software on the second computer upon receipt of the acceptance input.

13. The system of claim 12, further comprising:
wherein the one or more computer readable media further include instructions stored thereon that when executed configure the one or more computers to:
execute, by the one or more computers, a health link between the health monitoring software on the first computer to the health monitoring software on the second computer.

14. The system of claim 13, further comprising:
wherein the rewards calculation comprises a first rewards calculation and a second rewards calculation;
wherein the one or more wellness milestones include a first wellness milestone and a second wellness milestone;
wherein the one or more computer readable media further include instructions stored thereon that when executed configure the one or more computers to:
execute, by the one or more processors, the first rewards calculation on the first computer using the first wellness milestone;
execute, by the one or more processors, the second rewards calculation on the second computer using the second wellness milestone.

15. The system of claim 14,
wherein the first rewards calculation includes the second wellness milestone.

16. The system of claim 15,
wherein the second rewards calculation includes the first wellness milestone.

17. The system of claim 2,
wherein the one or more rewards include one or more of changes in life insurance policy benefits, cash back on credit card purchases, exclusive offers from businesses, changes in credit limits, changes in credit interest rates, changes in health insurance rates, changes in auto insurance rates, and discounts on athletic accessories.

18. The system of claim 2,
wherein the one or more measurements include one or more measurements of exercise, heart rate, blood oxygen level, sleep time, body fat percentage, weight, flexibility, driving habits, food purchases, equipment purchases, and online activity.
